(19) United States
(12) Patent Application Publication
(10) Pub. No.: US 2024/0225069 A1
(43) Pub. Date: Jul. 11, 2024

(54) METHOD AND APPARATUS TO FORM A MYCELIUM-BASED FOOD PRODUCT

(71) Applicant: MyForest Foods Co., Green Island, NY (US)

(72) Inventors: Russell Allan Hazen, Clifton Park, NY (US); Jacob Tyler Meyers, Ballston Spa, NY (US); Jeff Andrew Mundt, Hackettstown, NJ (US)

(21) Appl. No.: 18/407,127

(22) Filed: Jan. 8, 2024

Related U.S. Application Data
(60) Provisional application No. 63/479,338, filed on Jan. 10, 2023.

(51) Int. Cl. A23L 31/00; A23L 29/00

(52) U.S. Cl. CPC A23L 31/00; A23L 29/04; A23L 29/045

(57) ABSTRACT

Methods for producing a mycelium-based food are disclosed herein. An aerial mycelium may be cultivated and processed into one or more aerial mycelium segments. The aerial mycelium segments may be placed in a vacuum tumbler and processed into the mycelium-based food. In some embodiments, processing may involve boiling the aerial mycelium segments, flavoring the aerial mycelium segments, and drying the aerial mycelium segments all within the vacuum tumbler.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims priority to U.S. Provisional App. No. 63/479338, the entirety of which is hereby incorporated by reference under 37 CFR 1.57.

### BACKGROUND

### Field

This application relates generally to edible mycelia suitable for use as food products or ingredients, methods of making edible mycelia and edible mycelial food products or ingredients, and in particular, to edible aerial mycelia and methods of making the same. Such a food product or ingredient can include edible aerial mycelium having a flavor and texture that is analogous to a whole-muscle, animal-based meat product, such as for example mycelium-based bacon, or other meat alternatives.

### Description

There is increasing demand for mycelium-based products in the food industry (for example, as an alternative, e.g., substitute, to an animal-based meat), as such products offer the potential for environmentally-friendly alternatives to currently-favored animal-based food products. Therefore, there remains a need for novel mycelium-based foods that can serve as animal-based meat alternatives and which can also offer unique sensory, nutritive, sustainability, and economic advantages in and of themselves. Given that such mycelium-based food products are relatively new to the industrial world, there is also a need for improved methods for growing and processing mycelium that are repeatable and energy efficient while providing high quality and quantity mycelium-based food products, as well as methods of producing mycelium-based food products that are more energy and resource efficient.

### SUMMARY

For purposes of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

In some embodiments, disclosed herein is a method for producing a mycelium-based food product. The method may include providing one or more aerial mycelium segments and processing the one or more aerial mycelium segments in a vacuum tumbler to form one or more corresponding processed aerial mycelium segments.

In some embodiments, processing the one or more aerial mycelium segments in the vacuum tumbler may include at least one of: boiling the one or more aerial mycelium segments, flavoring the one or more aerial mycelium segments, and drying the one or more aerial mycelium segments.

In some embodiments, flavoring the one or more aerial mycelium segments may include contacting the one or more aerial mycelium segments with one or more flavoring components in the vacuum tumbler at a flavoring pressure.

In some embodiments, the one or more flavoring components may include one or more of: a brining fluid and a marinating fluid.

In some embodiments, the one or more flavoring components may comprise thiamine.

In some embodiments, the thiamine may be added based on a weight of the one or more aerial mycelium segments.

In some embodiments, the thiamine may be added in an amount equal to about 1/1,000th of the weight to the one or more aerial mycelium segments.

In some embodiments, the thiamine may be added based on a sugar content of the one or more aerial mycelium segments.

In some embodiments. the thiamine may be added in an amount between about ⅕th of a weight of the sugar in the one or more aerial mycelium segments to an amount equal to the weight of the sugar in the one or more aerial mycelium segments.

In some embodiments, contacting the one or more aerial mycelium segments with the one or more flavoring components in the vacuum tumbler may include: contacting the one or more aerial mycelium segments with a first flavoring component at a first flavoring pressure; and contacting the one or more aerial mycelium segments with a second flavoring component at a second flavoring pressure. The first flavoring pressure may be the same as or different from the second flavoring pressure.

In some embodiments, a volume of the one or more flavoring components may be proportional to a volume of the one or more aerial mycelium segments.

In some embodiments, the volume of the one or more flavoring components may be equal to between two to four times the volume of the one or more aerial mycelium segments.

In some embodiments, the flavoring pressure may be between about 0 mbar and about 1013.25 mbar.

In some embodiments, the flavoring pressure may be between about 0 mbar and 100 mbar.

In some embodiments, the flavoring pressure may be between about 30 mbar and about 45 mbar.

In some embodiments, the flavoring pressure may be between about 26 millimeters of mercury (mmHg) to about 33 mmHg.

In some embodiments, the one or more aerial mycelium segments may be contacted with the one or more flavoring components at a target temperature. The target temperature may be at or below 40° F.

In some embodiments, flavoring the one or more aerial mycelium segment may include mixing the one or more aerial mycelium segments and the one or more flavoring components.

In some embodiments, boiling may include boiling the one or more aerial mycelium segments in an aqueous solution for a boiling time.

In some embodiments, the boiling time may be about 20 minutes.

In some embodiments, boiling may include boiling the one or more aerial mycelium segments in an aqueous solution until the one or more aerial mycelium segments reach a target temperature.

In some embodiments the target internal temperature may be between about 135° F. and about 212° F.

In some embodiments the target internal temperature may be between about 165° F. and about 190° F.

In some embodiments the target internal temperature may be about 165° F.

In some embodiments, drying the one or more aerial mycelium may include curing the one or more aerial mycelium segments in the vacuum tumbler at a drying pressure and a drying temperature for a drying time.

In some embodiments, the drying pressure may be between about 3 and about 50 mbar.

In some embodiments, the drying temperature may be between about 40° F. and about 70° F.

In some embodiments, the drying time may be between 15 minutes and 120 minutes.

In some embodiments, curing the one or more aerial mycelium may include introducing air into the vacuum tumbler.

In some embodiments, the air may be ambient air.

In some embodiments, the air may be a preconditioned air with a target humidity between about 0% and 30% relative humidity.

In some embodiments, the method may also include removing the air from the vacuum tumbler to return the vacuum tumbler to a flavoring pressure.

In some embodiments, flavoring may include fatting the one or more aerial mycelium segments with a fat. The fat may be selected from the group consisting of an almond oil, an animal fat, an avocado oil, a butter, a canola oil , a coconut oil, a corn oil, a grapeseed oil, a hempseed oil, a lard, a mustard oil, an olive oil, a palm oil, a peanut oil, a rice bran oil, a safflower oil, a soybean oil, a sunflower seed oil, a vegetable oil, a vegetable shortening or a combination thereof.

In some embodiments, the method may include fatting the one or more processed aerial mycelium segments with a fat. The fat may be selected from the group consisting of an almond oil, an animal fat, an avocado oil, a butter, a canola oil , a coconut oil, a corn oil, a grapeseed oil, a hempseed oil, a lard, a mustard oil, an olive oil, a palm oil, a peanut oil, a rice bran oil, a safflower oil, a soybean oil, a sunflower seed oil, a vegetable oil, a vegetable shortening or a combination thereof.

According to some embodiments, disclosed herein is a method for producing a mycelium-based food product, such as of one or more segments. The method may include providing one or more aerial mycelium segments; flavoring the one or more aerial mycelium segments in a vacuum chamber at a flavoring pressure; and curing the one or more aerial mycelium in the vacuum chamber at a drying pressure and a drying temperature to form one or more corresponding processed aerial mycelium segments.

In some embodiments, disclosed herein is a system for producing a mycelium-based food product. The system may include one or more aerial mycelium segments and a vacuum tumbler. The vacuum tumbler may include a body containing the one or more aerial mycelium segments and a processor configured to form one or more corresponding processed aerial mycelium segments from the one or more aerial mycelium segments.

According to some embodiments, disclosed herein is a method for producing a mycelium-based food product. The method may include providing an aerial mycelium and processing the aerial mycelium in a vacuum tumbler to form a corresponding processed aerial mycelium.

### BRIEF DESCRIPTION OF THE DRAWINGS

The features and advantages of the methods and compositions described herein will become more fully apparent from the following description and appended claims, taken in conjunction with the accompanying drawings. These drawings depict only several embodiments in accordance with the disclosure and are not to be considered limiting of their scope. In the drawings, similar reference numbers or symbols typically identify similar components, unless context dictates otherwise. In some instances, the drawings may not be drawn to scale.

FIG. 1A illustrates an embodiment of a growth matrix suitable to support extra-particle aerial mycelial growth.

FIG. 1B illustrates an embodiment of extra-particle aerial mycelial growth extending from the growth matrix of FIG. 1A.

FIG. 1C illustrates an embodiment in which the extra-particle aerial mycelial growth and growth matrix in FIG. 1B have been divided to form a separated aerial mycelium and depleted growth matrix.

FIG. 1D illustrates an embodiment of the separated aerial mycelium in FIG. 1C that has been cut to form an aerial mycelium panel.

FIG. 2 illustrates an embodiment of a process for making an edible mycelium-based food product, such as a mycelium-based bacon.

FIG. 3 illustrates an embodiment of a process for making an edible mycelium-based food product, such as a mycelium-based bacon.

FIG. 4 illustrates an embodiment of a vacuum tumbler configured to process one or more aerial mycelium segments.

### DETAILED DESCRIPTION

While the mycelium-based food industry and production systems are still in their infancy, challenges for food production with mycelium have already been observed. For instance, while certain flavoring and curing techniques have regularly been used for animal-based meats, the cellular makeup and textural consistency of mycelium-based meats have precluded use of similar flavoring and curing methods for mycelium. For instance, mycelium tends to be hydrophobic in nature, which may prevent the ready absorption of water-based seasoning/marinades into the material's structure for enhanced flavoring. Even when a marinade is applied to mycelium material, the time necessary to accomplish acceptable flavorant penetration is often impractical by industrial food-production standards. There is therefore a need in mycelium-based food production, for flavoring techniques which overcome the natural hydrophobic attributes of the mycelium-based material itself.

In order to increase the impact of flavorants/marinades on animal-based meats, injection techniques have often been used to facilitate penetration of flavorants into animal tissue or areas between an animal's tissue and skin. Such injections of flavorants, which may be an acceptable technique for certain animal-based meats, have the potential for actually altering the physical structure of a mycelium material, creating holes within the product which do not readily close up (upon withdrawal of the injector), since such mycelium material also tends to be inelastic in nature at the cellular level. Therefore, there is a further need for flavoring techniques which allow for the rapid and pervasive uptake of flavorants within mycelium material itself, without creating the internal cavities that flavorant injections would create, were such techniques to be used.

The present application is generally directed toward systems and methods for growing an aerial mycelium (biomaterial) and processing the aerial mycelium into mycelium-based food products, such as a mycelium-based bacon or a mycelium-based jerky. As described in greater detail below, an aerial mycelium may be cultured and preprocessed into one or more aerial mycelium segments. These aerial mycelium segments can comprise any extra-particle aerial mycelial growth that has been removed from a growth matrix or a portion thereof. The aerial mycelium segments may be processed in a vacuum chamber, such as a vacuum tumbler, to produce the mycelium-based food product. Processing the aerial mycelium segments in the vacuum chamber may include boiling the segments, flavoring the segments, drying the segments, and/or other processing steps. For example, the segments may be boiled, soaked in a brining fluid in a vacuum, and subsequently cured in a vacuum of the same apparatus, such that flavorants may fully penetrate mycelial tissue without damage to the physical structure (that would occur from use of traditional techniques such as flavor injection), and without interference from the inherent hydrophobic nature of the material. While such processing can also take place in numerous devices in a particular sequence (which are also contemplated by this disclosure), the advantage of using a singular device such as a single, multi-functional vacuum tumbler, include rapid penetration of flavorants into uniquely problematic food tissue, in an expedited time frame, with reduced industrial resources.

Advantageously, methods and systems described herein create a more flavorful, succulent mycelium-based food product than was previously available. Moreover, the processes and systems require substantially (e.g., exponentially) less time to produce a more delectable mycelium-based food product such that mycelium-based foods can be produced in a matter of hours (or potentially less), rather than days, weeks, or even months required by prior art solutions such as only injecting a marinade or applying a marinade to the mycelial tissue in an ambient environment. Furthermore, the systems and methods produce a tempting mycelium-based alternative that more closely mimics other animal-based foods than previously possible, such that the difference between the traditional animal-based food product and the alternative mycelium-based food product is nearly imperceptible.

In some aspects, using a vacuum on the mycelium-based biomaterial during one or more of the processing steps can also result in a favorable change in texture to the product. Using the vacuum during processing can remove the intrinsic air within the mycelium-based biomaterial, which can increase the density of the resulting product. Using the vacuum during processing can enhance the natural fiber structure within the mycelium. The natural fiber structure may be enhanced while maintaining the growth grain of the aerial mycelium. Using the vacuum during processing can allow more flavorants (e.g., salt and sugar) to remain within the product. Using the vacuum during processing can have a significant impact on the protein structure morphology, by denaturing the proteins and changing the resulting product texture. During a vacuum drying step, moisture from the product can be removed from the center of the product, thus drying the product from the “inside out,” rather than a conventional process like an oven or dehydrator where the temperatures are higher and the product dries from the surface in. Potentially undesirable fungal flavors (e.g., bitterness or other traditional mushroom-like flavors) of the products made using the vacuum processes herein, may be lower relative to non-vacuum processes production processes, because the potentially undesirable flavors can be more efficiently removed through boil and vacuum steps conducted in a particular sequence and/or within a singular device, develop less due to lower drying temperatures, or are simply less noticeable due to better, stronger introduction and retention of other desirable flavors or odor particle replacement through such processes.

### Definitions

“Mycelium” as used herein refers to a connective network of fungal hyphae, with mycelia being the plural form of mycelium.

“Hyphae” as used herein refers to branched filament vegetative cellular structures that are interwoven to form mycelium.

“Mycelium-based” as used herein refers to a composition substantially comprising mycelium.

“Substrate” as used herein refers to a material suitable for supporting the growth of an aerial mycelium of the present disclosure. In some embodiments, the substrate is a natural substrate.

“Growth media” or “growth medium” as used herein refers to a matrix containing a substrate and an optional further source of nutrition wherein the substrate and/or the nutrition source are intended for fungal consumption to support mycelial growth.

“Growth matrix” as used herein refers to a matrix containing a growth medium and a fungus. In some embodiments, the fungus is provided as a fungal inoculum; thus, in such embodiments, the growth matrix comprises a fungal-inoculated growth medium. In other embodiments, the growth matrix comprises a colonized substrate.

“Colonized substrate” as used herein refers to an inoculated substrate that has been incubated for sufficient time to allow for fungal colonization. A colonized substrate of the present disclosure can be characterized as a contiguous hyphal mass grown throughout the entirety of the volume of the growth media substrate.

“Air,” “air content” and “airflow” as used herein can refer to various compositions of various gases, and flow of those gases suitable for the products and methods described herein, and should not otherwise be limited to a particular composition or ratio of gases.

“Appressed mycelium” as used herein refers to a continuous mycelium obtained from extra-particle appressed mycelial growth, and which is substantially free of growth matrix.

“Aerial mycelium” as used herein refers to mycelium obtained from extra-particle aerial mycelial growth, and which is substantially free of growth matrix.

“Extra-particle mycelial growth” (EPM) as used herein refers to mycelial growth, which can be either appressed or aerial.

“Extra-particle aerial mycelial growth” as used herein refers to a distinct mycelial growth that occurs away from and outward from the surface of a growth matrix. Extra-particle aerial mycelial growth can exhibit negative gravitropism. In a geometrically unrestricted scenario, extra-particle aerial mycelial growth could be described as being positively gravitropic, or neutrally gravitropic, aerial, and radial in which growth will expand in all directions from its point source.

“Extra-particle appressed mycelial growth” as used herein refers to a distinct mycelial growth that is surface-tracking (thigmotropic), is determinate in growth substantially orthogonal to the surface of a growth matrix, is indeterminate in growth substantially parallel to the surface of the growth matrix. Extra-particle appressed mycelial growth can exhibit positive gravitropism.

“Fruiting body” as used herein refers to a stipe, pileus, gill, pore structure, or a combination thereof.

“Determinate growth” as used herein refers to growth that occurs until a maximum final dimension is achieved while growth continues to occur in other dimensions. Either determinate or indeterminate mycelial growth above the surface of a growth matrix defines a mycelium's native thickness.

“Indeterminate growth” as used herein refers to growth that expands indefinitely in a given direction as long as mycelial growth is occurring.

“Positive gravitropism” as used herein refers to growth that preferentially occurs in the direction of gravity.

“Negative gravitropism” as used herein refers to mycelial growth that preferentially occurs in the direction away from gravity. As disclosed herein, extra-particle aerial mycelial growth can exhibit negative gravitropism. Without being bound by any particular theory, this may be attributable at least in part to the geometric restriction of the growth format, such as a growth format where an uncovered tool (or tray) having a bottom and side walls contains a growth matrix. With such geometric restriction, growth will primarily occur along the unrestricted dimension(s), which in the scenario is primarily vertically (negatively gravitropic) since the side and bottom walls restrict growth in those directions.

“Compressing aerial mycelium” or “compressed aerial mycelium” as used herein refers to the increase of aerial mycelium density, as a result of reducing aerial mycelium volume (i.e., reducing the length, width, and/or thickness) by applying a selected pressure to the aerial mycelium. It will be understood that the density of the compressed material as used herein is the steady state density after any rebound that occurs after the initial compression is applied to and released from the material. In some embodiments, the volume of the aerial mycelium is reduced after it has been detached from the growth matrix, to increase aerial mycelium density. In some further embodiments, the compressed aerial mycelium has a mean density, wherein the mean density of the compressed aerial mycelium is between 0.5 g/cm³ to 0.8 g/cm³.

A “native” property as used herein refers to a property associated with a mycelium obtained after an incubation time period has elapsed and upon subsequent removal of the mycelial growth from a growth matrix, and prior to any optional environmental, physical, or other post-processing step(s) or excursion(s), whether intentional or unintentional, that substantially alters the property.

### Definitions and Properties Related to Edibility

In some aspects, the present disclosure provides for an edible mycelium-based food product or an edible mycelium-based food ingredient.

“Edible” as used herein refers to being generally regarded as safe to be eaten by humans or pets, especially after cooking; being generally considered palatable by humans or pets; and/or being capable of being substantially masticated by humans or pets.

An edible mycelium-based food product or food ingredient can be distinguished from a mycelium-based medicine or from a mycelium-based nutritional supplement upon consideration of factors such as the method, form and/or quantity for ingestion.

In some embodiments, an edible mycelium-based product or ingredient of the present disclosure can exclude a mycelium-based medicine. In some other embodiments, an edible mycelium-based product or ingredient of the present disclosure can exclude a mycelium-based nutritional supplement.

In some aspects, the present disclosure provides for an aerial mycelium characterized by its native nutritional content. As used herein, “native nutritional content” refers to the nutritional content of an aerial mycelium obtained after an incubation time period has elapsed and the resulting mycelial growth has been removed from a growth matrix, and prior to performing any optional environmental, physical, or other post-processing step(s) that may substantially alter the nutritional content of the aerial mycelium so obtained. Non-limiting examples of native nutritional content include native protein content, native fat content, native carbohydrate content, native dietary fiber content, native vitamin content, native mineral content, and so on. Typically, the nutritional content is reported based on the dry weight of the mycelium.

Thus, in some aspects, an aerial mycelium of the present disclosure is characterized as having a native protein content. In some embodiments, an aerial mycelium of the present disclosure is characterized as having a native protein content of at least about 20% (w/w), or at least about 25% (w/w), on a dry weight basis. In some further embodiments, an aerial mycelium of the present disclosure is characterized as having a native protein content of at most about 50% (w/w), or at most about 45% (w/w), on a dry weight basis. In some embodiments, an aerial mycelium of the present disclosure is characterized as having a native protein content within a range of about 20% to about 50% (w/w), about 21% to about 49% (w/w), about 22% to about 48% (w/w), about 23% to about 47%, about 24% to about 46% (w/w), about 25% to about 45% (w/w), about 26% to about 44% (w/w), about 27% to about 43% (w/w) or about 28% to about 42% (w/w), on a dry weight basis. In some more particular embodiments, an aerial mycelium of the present disclosure is characterized as having a native protein content of about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w) or about 50% (w/w), on a dry weight basis.

In some aspects, an aerial mycelium of the present disclosure is characterized as having a native fat content. As used herein, native fat content refers to native triglyceride content, and can be determined according to methods known to persons of ordinary skill in the art. In some embodiments, an aerial mycelium of the present disclosure is characterized as having a native fat content of at most about 7% (w/w), or at most about 6% (w/w), on a dry weight basis. In some further embodiments, an aerial mycelium of the present disclosure is characterized as having a native fat content of at least about 1% (w/w), at least about 1.5% (w/w), at least about 2% (w/w), at least about 2.5% w/w) or at least about 3% (w/w), on a dry weight basis. In yet some further embodiments, an aerial mycelium of the present disclosure is characterized as having a native fat content within a range of about 1% (w/w) to about 7% (w/w), or about 1.5% to about 6.5% (w/w), on a dry weight basis. In some more particular embodiments, an aerial mycelium of the present disclosure is characterized as having a native fat content of about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.1% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w),about 5.6% (w/w), about 5.7% (w/w), about 5.8% (w/w), about 5.9% (w/w), about 6.0% (w/w), about 6.1% (w/w), about 6.2% (w/w), about 6.3% (w/w), about 6.4% (w/w), about 6.5% (w/w), about 6.6% (w/w), about 6.7% (w/w), about 6.8% (w/w), about 6.9% (w/w) or about 7.0% (w/w), on a dry weight basis.

In some aspects, an aerial mycelium of the present disclosure is characterized as having a native carbohydrate content. In some embodiments, an aerial mycelium of the present disclosure is characterized as having a native carbohydrate content of at least about 30% (w/w), or at least about 35% (w/w), on a dry weight basis. In some further embodiments, an aerial mycelium of the present disclosure is characterized as having a native carbohydrate content of at most about 60% (w/w), or at most about 55% (w/w), on a dry weight basis. In some embodiments, an aerial mycelium of the present disclosure is characterized as having a native carbohydrate content within a range of about 30% (w/w) to about 60% (w/w), about 35% (w/w) to about 55% (w/w), about 40% (w/w) to about 55% (w/w), about 40% (w/w) to about 50% (w/w), or about 45% (w/w) to about 55% (w/w), on a dry weight basis. In some more particular embodiments, an aerial mycelium of the present disclosure is characterized as having a native carbohydrate content of about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w), about 50% (w/w), about 51% (w/w), about 52% (w/w), about 53% (w/w), about 54% (w/w), about 55% (w/w), about 56% (w/w), about 57% (w/w), about 58% (w/w), about 59% (w/w) or about 60% (w/w), on a dry weight basis.

In some aspects, an aerial mycelium of the present disclosure is characterized as having a native inorganic content. As used herein, native inorganic content is reported based on ash content, which can be determined according to methods known to persons of ordinary skill in the art. In some embodiments, an aerial mycelium of the present disclosure is characterized as having a native inorganic content of at least about 5% (w/w), at least about 6% (w/w), at least about 7% (w/w), at least about 8% (w/w) or at least about 9% (w/w), or at least about 10% (w/w), on a dry weight basis. In some further embodiments, an aerial mycelium of the present disclosure is characterized as having a native inorganic content of at most about 20% (w/w), on a dry weight basis. In some embodiments, an aerial mycelium of the present disclosure is characterized as having a native inorganic content within a range of about 5% (w/w) to about 20% (w/w), about 6% (w/w) to about 20% (w/w), about 7% (w/w) to about 20% (w/w), about 8% (w/w) to about 20% (w/w), about 9% (w/w) to about 20% (w/w), about 10% (w/w) to about 20% (w/w), or about 9% (w/w) to about 18% (w/w), on a dry weight basis. In some more particular embodiments, an aerial mycelium of the present disclosure is characterized as having a native inorganic content of about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w) or about 20% (w/w), on a dry weight basis.

In some aspects, an aerial mycelium of the present disclosure is characterized as having a native dietary fiber content. In some embodiments, an aerial mycelium of the present disclosure is characterized as having a native dietary fiber content of at least about 15% (w/w), on a dry weight basis. In some further embodiments, an aerial mycelium of the present disclosure is characterized as having a native dietary fiber content of at most about 35% (w/w), on a dry weight basis. In some embodiments, an aerial mycelium of the present disclosure is characterized as having a native dietary fiber content within a range of about 15% (w/w) to about 35% (w/w), on a dry weight basis. In some more particular embodiments, an aerial mycelium of the present disclosure is characterized as having a native dietary fiber content of about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 31% (w/w), about 32% (w/w), about 33% (w/w), about 34% (w/w) or about 35% (w/w), on a dry weight basis.

In some aspects, the present disclosure provides for an aerial mycelium having a native potassium content of at least about 4000 milligrams of potassium per 100 grams of dry aerial mycelium. In some embodiments, an aerial mycelium of the present disclosure has a native potassium content within a range of about 4000 mg potassium per 100 g dry aerial mycelium to about 7000 mg potassium per 100 g dry aerial mycelium. In some further embodiments, an aerial mycelium of the present disclosure has a native potassium content within a range of about 4500 mg potassium per 100 g dry aerial mycelium to about 6500 mg potassium per 100 g dry aerial mycelium.

### Methods Related to Edibility

Various methods can be implemented to provide varying properties within different portions of an aerial mycelium, to mimic “marbling” or other desired characteristics of meat products. For example, some methods can implement property-affecting organisms during the incubation and/or growth cycle to provide a heterogenous edible aerial mycelium for different food alternatives. Other step(s) that can provide heterogenous characteristics include selective hardening, freezing, fracturing, boiling, brining, drying, and/or other step(s).

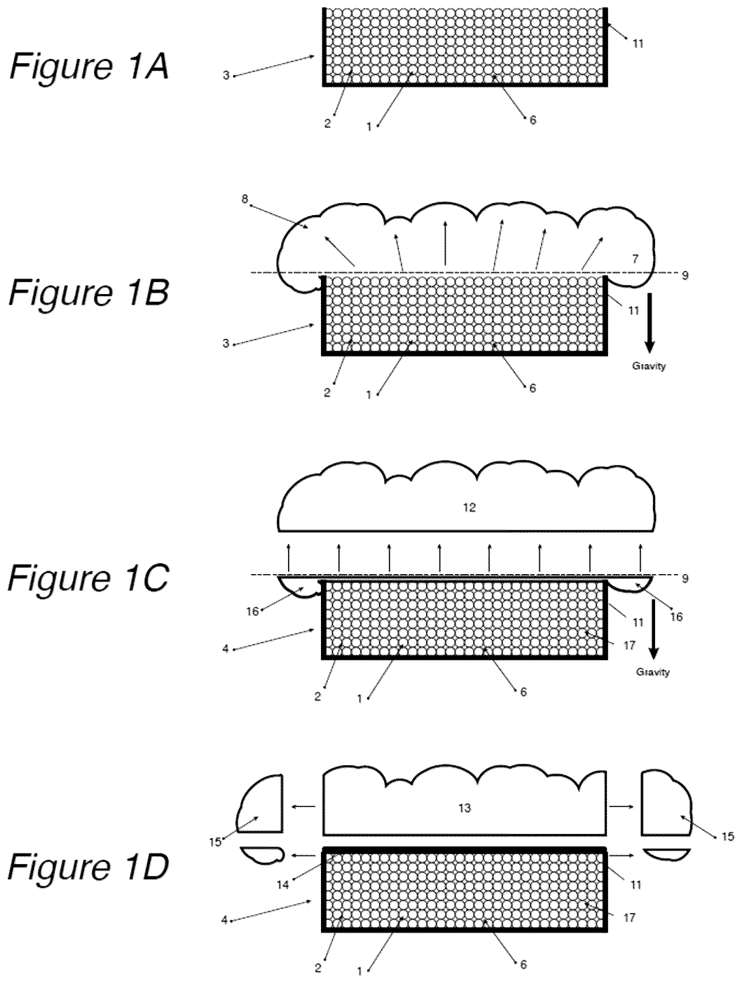

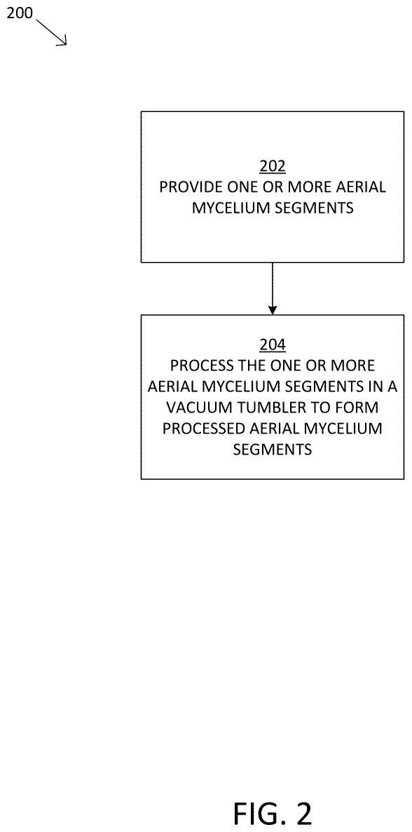

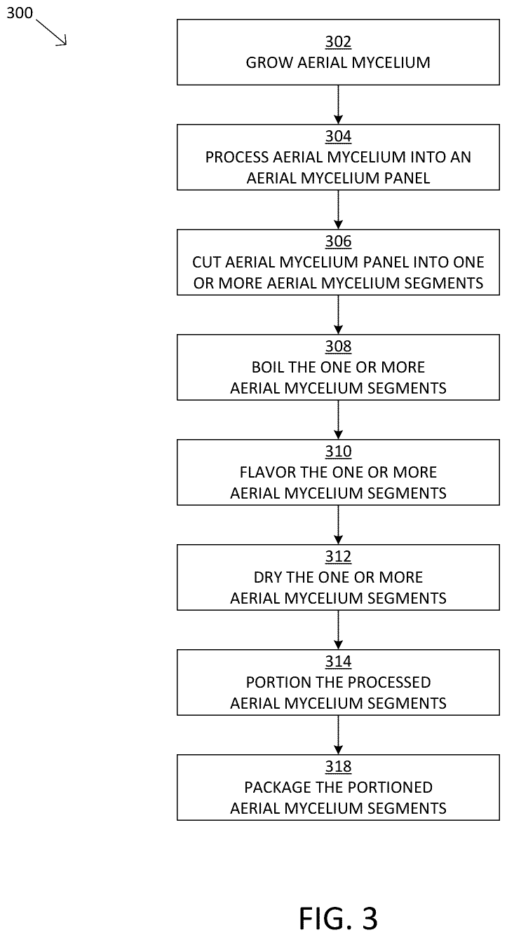

In some aspects, the present disclosure provides for methods of post-processing an aerial mycelium of the present disclosure. Methods of post-processing as described herein can be used to modify a mycelium, including an aerial mycelium, to provide an edible food ingredient scaffold that mimics an animal-based food product, such as mycelium-based bacon. It is contemplated that other shapes of scaffolding can be produced to help facilitate creating the traditional appearance of an animal-based food product, such as a shrimp or a fish-based product (i.e. smoked fish). As another example, scaffolding can be designed to produce patty shapes or elongated ovular shapes (following additional process steps) for mimicking the appearance of fish or seafood patties, the unique configuration of elongated gefilte fish, or the appearance of other traditional meat-based cultural foods, such as kibbeh. This post-processing can include steps such as cutting, slicing, extruding, pressing, and/or perforating. It will be understood that one or more of these post-processing steps can be implemented to produce a desired shape for a mycelium (e.g., aerial mycelium, panel, segment) but before the mycelium is processed in a vacuum chamber. For example, a post-processing step can be implemented after a separated aerial mycelium is formed (e.g., FIG. 1C below) to produce an aerial mycelium with the desired shape. In some embodiments, substantially the same desired shape can be maintained within and/or after additional post-processing steps. The growth grain of the aerial mycelium may also be maintained within and/or after the additional post-processing steps. This can advantageously avoid or reduces the need for molding a product shape, or implementing other post-vacuum chamber shape-formation processes. The post-processing can include amending the mycelium (e.g., with, or in some embodiments, without changing its desired shape) through boiling, brining, drying, fatting, flavoring, marinating, and/or the incorporation of additives. The post-processing of the mycelium provides a mycelium-based product that more closely resembles animal tissue. Any number of steps or combinations of steps can be performed in any variety of sequences to achieve the desired result, separately from or in combination with the other methods and steps herein, such as those described with reference to FIGS. 2-3 below. Additional details and/or other methods of processing mycelial tissue are disclosed in US2020/0024557A1 and PCT Pub. No. WO2022/235694, the entire contents of which are hereby incorporated by reference in their entirety to the extent not inconsistent with the content of this disclosure.

### Additives

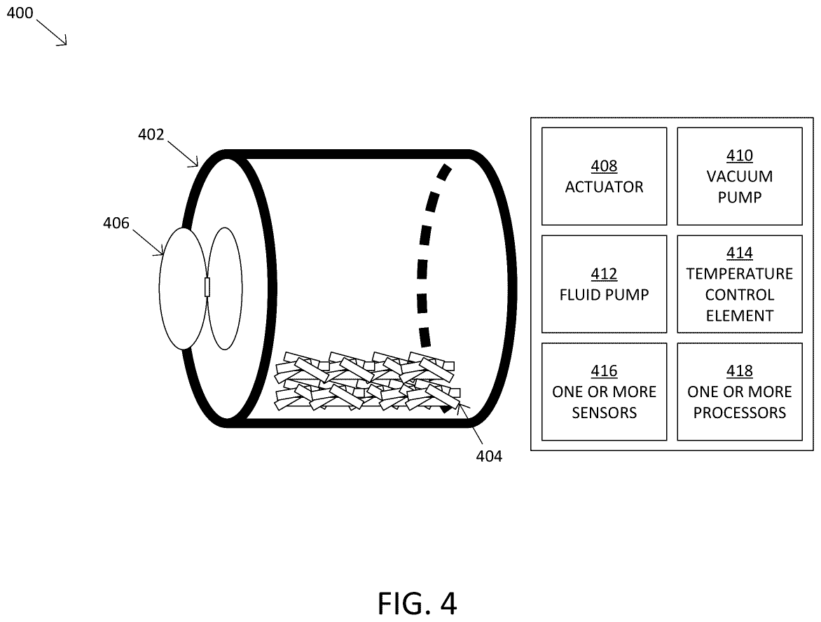

In some aspects, the present disclosure provides for the incorporation of one or more additives into the mycelial tissue or onto the surface of the mycelial tissue. The additive(s) can be incorporated during or after the growth of the mycelium, and/or before, during or after any one or more post-processing steps, such as the steps in the methods described with reference to FIGS. 2 and 3, and with the apparatus in FIG. 4.

Useful additives for incorporation into edible mycelia of the present disclosure, can include additives that are commonly found in Worcestershire-imitating sauces, soy sauces, fish-flavoring imitating sauces, vinegars, and smoke-flavoring sauces. In some aspects, an additive can be a fat, a protein, a peptide, an amino acid, a flavorant, an aromatic agent, a mineral, a vitamin, a micronutrient, a colorant, or a preservative; or a combination thereof. An additive can be a naturally occurring additive or an artificial additive, or a combination thereof.

Non-limiting examples of a fat include almond oil, animal fat, avocado oil, butter, canola oil (rapeseed oil), coconut oil, corn oil, grapeseed oil, hempseed oil, lard, mustard oil, olive oil, palm oil, peanut oil, rice bran oil, safflower oil, soybean oil, sunflower seed oil, vegetable oil, or vegetable shortening; or a combination thereof. In some embodiments, the fat is a plant-based oil or fat. In some embodiments, the plant-based oil is coconut oil or avocado oil. In some embodiments, the oil is a refined oil. In some embodiments, the fat is animal fat. In some embodiments, the animal fat is pork fat, chicken fat or duck fat.

Non-limiting examples of a flavorant include a smoke flavorant, umami, maple, a salt, a sweetener, a spice, or a meat flavor (e.g., pork flavor); or a combination thereof. Non-limiting examples of a smoke flavorant include applewood flavor, hickory flavor, liquid smoke; or a combination thereof.

Non-limiting examples of umami include a glutamate, such as sodium glutamate.

Non-limiting examples of a salt include sodium chloride, table salt, flaked salt, sea salt, rock salt, kosher salt, or Himalayan salt; or a combination thereof.

Non-limiting examples of a sweetener include sugar, cane sugar, brown sugar, honey, molasses, juice, nectar, or syrup (e.g., maple syrup), saccharin, aspartame, acesulfame potassium (Ace-K), sucralose, neotame, advantame, steviol glycosides (extracts of Stevia rebaudiana), and extracts obtained from Siraitia grosvenorii Swingle fruit, also known as Luo Han Guo or monk fruit; or a combination thereof.

Non-limiting examples of a colorant include beet extract, beet juice, or paprika; or a combination thereof.

Non-limiting examples of a spice include paprika, pepper, mustard, garlic, chili, jalapeno, and the like; or a combination thereof.

A non-limiting example of an aromatic agent having a distinctive fragrance include allicin.

Non-limiting examples of a mineral include iron, magnesium, manganese, selenium, zinc, calcium, sodium, potassium, molybdenum, iodine, or phosphorus; or a combination thereof.

Non-limiting examples of a vitamin include ascorbic acid (vitamin C), biotin, a retinoid, a carotene, vitamin A, thiamine (vitamin B1), riboflavin (vitamin B2), niacin (vitamin B3), pantothenic acid (vitamin B5), pyridoxine (vitamin B6), folate, folic acid (vitamin B9), cobalamin (vitamin B12), choline, calciferol (vitamin D), alpha-tocopherol (vitamin E) or phylloquinone (menadione, vitamin K); or a combination thereof.

Non-limiting examples of a protein include a plant-derived protein, a heme protein; or a combination thereof.

Non-limiting examples of an amino acid include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine; or a combination thereof.

One or more additives can be incorporated into a mycelium of the present disclosure at virtually any step(s) during or between the mycelium growth or post-processing steps described herein.

In some embodiments, one or more additives can be included in (e.g., admixed with) a growth matrix, growth media, growth media substrate, and/or in a further source of nutrition (e.g., a nutritional supplement) in the growth media.

As disclosed in US2020/0024557A1, an additive can be deposited on the growth media during the growth process, either through liquid or solid deposition, or though natural cellular uptake (bioadsorption), e.g., increasing mineral content in the growth media, to increase final content in the panel of tissue. Furthermore, during growth, desired nutrients, flavors, or other additives can be aerosolized into the growth chamber, condense on the propagating tissue, and be incorporated into the matrix.

As further disclosed in US2020/0024557A1, a grown mycelial panel can be infused with at least one additive.

In some embodiments, one or more additives is added to a mycelium during the incubation time period. In some embodiments, one or more additives is added to a mycelium after the incubation time period. In some embodiments, one or more additives is added to a mycelium after extraction from the growth matrix.

In some embodiments, one or more additives is added during one or more post-processing steps. Thus, one or more additives can be incorporated into a mycelium by injection into a mycelium (although this may not be preferred), during boiling (e.g., by incorporating additives in the aqueous solution used for boiling), during brining (e.g., in a brine fluid), during fatting (e.g., in the fat), or at any time prior to packaging. An additive can be included with the packaged goods.

An edible mycelium of the present disclosure, in any form, including an aerial mycelium for use as a food ingredient, a food product, a strip of mycelium-based bacon, strips of mycelium-based brisket, corned-beef, pastrami or other deli alternatives and the like, aerial mycelium-based jerky, can be packaged to provide a finished product. The package can include a label describing cooking instructions, storage or handling instructions, nutritional information, or a combination thereof.

Thus, in some embodiments, there is provided a mycelium-based bacon product, said product comprising oyster mushroom mycelium, coconut oil, organic sugar, sea salt, vegan natural flavors and beet juice. The oyster mushroom mycelium can be obtained from an aerial mycelial of the present disclosure, wherein the aerial mycelium is a growth product of the fungus Pleurotus ostreatus, and wherein the aerial mycelium has a growth grain. An aerial mycelium can be characterized by its direction of mycelial growth. The “growth grain” of an aerial mycelium may be visible as a function of aggregations of hyphae that are oriented into larger aligned structures. The growth grain may be generally aligned along a first axis, which may be referred to herein as an “aerial mycelial growth axis,” such as approximately perpendicular to a substrate as shown in FIG. 1B. The orientation of the growth grain may be evident at a macroscopic scale. The orientation of the growth grain may be made more evident by the ease with which an aerial mycelium panel tears along this growth grain, in analogy to the grain of a cut of an animal-based meat. Other methods for culturing mycelium, such as liquid fermentation which creates an amorphous mycelial material, do not produce a mycelium with such a growth grain.

In some embodiments, the product is not an extruded product and is not a minced product. The product can be packaged, and the package can include a label comprising cooking instructions, storage or handling instructions, nutritional information, or a combination thereof.

Aerial mycelia of the present disclosure, and methods of making and/or processing aerial mycelia of the present disclosure, can be adapted to prepare a variety of food products, including whole-muscle animal-based meat alternatives, seafood alternatives, poultry alternatives and carbohydrate-based food alternatives. Additional non-limiting examples of food products that can be prepared from aerial mycelia of the present disclosure include a bacon alternative, a jerky alternative, a deli meat alternative, a steak alternative, a chicken alternative, a chicken nugget alternative, a fish filet alternative, a shellfish alternative, a clam alternative, an oyster alternative, a scallop alternative, a shrimp alternative, a smoked salmon alternative, a pulled pork alternative, a cheese alternative, a convenience food, a snack food, a pasta, a confection, a bread or a baked good.

### Hardening

It may be advantageous to harden a portion of an edible aerial mycelium panel or an aerial mycelium segment or segments, for example, to provide certain characteristics and/or edible food alternatives. Thus, in some embodiments, a method of processing an edible aerial mycelium can include providing a panel or segment(s) comprising an edible aerial mycelium; and selectively hardening at least a portion of an outer surface of the panel or segment(s), relative to a remainder of the panel or segment(s). In some embodiments, substantially the entirety of the exposed outer surface of the panel or segment(s) can be hardened.

The hardening can be provided in different ways. For example, the hardening can comprise drying at least a portion of the outer surface. Drying can comprise drying by at least one of radiation drying, convection drying, conduction drying, freeze drying, microwave drying, evaporative drying, and vacuum drying, one or more of which could be accomplished via a vacuum tumbler. The radiation drying can comprise infrared radiation drying. Such drying steps can also be implemented for reasons other than hardening, for example, within a vacuum vessel (e.g., vacuum tumbler).

A hardened panel or segment(s) can be further processed through fracturing steps. For example, the method can comprise fracturing the at least the portion of the outer surface from a core and a base of the panel or segment(s). This can disconnect the hyphae of the panel from one surface, while keeping the core and the base bound, to slice, without collapsing the structure. Fracturing can comprise mechanical shearing, such as shearing with a compressed air cutter, a knife, and a fluted roller. Other fracturing step(s) described further herein can be implemented with the hardening step(s), as well as freezing, boiling, brining, further drying, and/or other step(s).

### Flash Freezing

It may be advantageous to flash freeze an aerial mycelium panel or an aerial mycelium segment, for example, to provide certain characteristics and/or edible food alternatives. Thus, in some embodiments, a method of processing an edible aerial mycelium can include providing a panel or segment(s) comprising an edible aerial mycelium, wherein the edible aerial mycelium comprises a growth grain; and flash freezing the panel or segment(s).

Different flash freezing methods can be implemented for various results. In some embodiments, flash freezing comprises exposing the panel or segment(s) to a temperature of less than or equal to about minus 120° C., of less than or equal to about minus 180° C., or of about minus 196° C. The flash freezing can comprise exposing the panel or segment(s) to at least one of a blast freezer, liquid nitrogen, and/or other methods.

The flash-freezing method can also include fracturing. For example, a natural fault line can extend along a portion of the growth grain, and the panel or segment(s) can be fragmented along the natural fault line. The fracturing can be performed similarly to other fracturing described herein. Other steps described further herein can be implemented with the flash freezing step(s), such as hardening, boiling, brining, drying, and/or other steps. In some embodiments herein, the growth grain and fault lines of the mycelium can be configured to advantageously and unexpectedly provide a “flakiness” akin to that of some non-mycelium food products, such as fish (e.g., a fish filet).

### Fracturing

It may be advantageous to flash freeze an aerial mycelium panel or an aerial mycelium segment, for example, to provide certain characteristics and/or edible food alternatives. For example, a method of processing an edible aerial mycelium can comprise providing a panel or segment(s) comprising an edible aerial mycelium, wherein the edible aerial mycelium comprises a growth grain, wherein at least one natural fault line extends along a portion of the growth grain; and fracturing the panel or segment(s) along the natural fault line.

The fracturing can provide a mycelium-based food product which can be a seafood alternative product, a poultry alternative product, a pork alternative product, and/or a beef alternative product. For example, fracturing may provide at least one of a scallop, smoked salmon, and/or chicken nugget alternative product, or other alternative products. In some embodiments, the fracturing can be performed creating approximately 1 inch diameter pieces (e.g., scallops), or other desired sizes.

Other steps described further herein can be implemented with the fracturing step(s), such as hardening, freezing, boiling, brining, drying, and/or other steps.

### Selective Size Cutting and Shaping

It may be advantageous to cut an aerial mycelium panel or segment(s) in desired ways, for example, to provide certain characteristics and/or edible food alternatives. This cutting process can be similar in some ways as those described herein with reference to bacon but can be advantageously different to provide other characteristics and/or edible food alternatives, such as a shrimp alternative.

For example, a method of processing an edible aerial mycelium can comprise providing a panel comprising an edible aerial mycelium, wherein the edible aerial mycelium comprises a growth grain; compressing at least a portion of the panel; and cutting at least a portion of the panel in a direction substantially parallel to the growth grain.

Cutting can further include cutting the panel to form at least one aerial mycelium segment. A thickness of the aerial mycelium segment can be at least about 1 inch, at least about 2 inches or at least about 3 inches. A plurality of segments can be formed that fall within a desired size and/or shape. For example, the plurality of segments can comprise a standard shrimp size. In some embodiments, the method can further comprise rolling the segments(s) into an approximately cylindrical shape. For example, rolling can comprise positioning the segment between two conveyors, each operating at a different speed relative to the other, to provide the rolling functionality. The cutting, rolling, and/or other processes can be implemented to provide one or more aerial mycelium panels and/or segments with a desired shape, as described elsewhere herein.

### Discussion Of The Figures

Aerial mycelia of the present disclosure, and methods of making and/or processing aerial mycelia of the present disclosure, can be adapted to prepare a variety of food products, including whole-muscle animal-based meat alternatives, seafood alternatives, poultry alternatives and carbohydrate-based food alternatives. Additional non-limiting examples of food products that can be prepared from aerial mycelia of the present disclosure include a bacon alternative, a jerky alternative, a deli meat alternative, a steak alternative, a chicken alternative, a chicken nugget alternative, a fish filet alternative, a shellfish alternative, a clam alternative, an oyster alternative, a scallop alternative, a shrimp alternative, a smoked salmon alternative, a pulled pork alternative, a cheese alternative, a convenience food, a snack food, a pasta, a confection, a bread or a baked good.

The following are examples of apparatus, systems and methods that can be similar as others disclosed herein, but with some differences, to provide aerial mycelia or products with different alternative characteristics. Not all of the steps or components are required (some are optional), and additional steps may be performed, or components can be implemented, relative to those listed for each example.

FIGS. 1A-1D demonstrate embodiments for culturing an extra-particle aerial mycelial growth, isolation of an aerial mycelium, and processing of the aerial mycelium into an aerial mycelium panel.

FIG. 1A illustrates an embodiment of a growth matrix 3 suitable to support extra-particle mycelial growth, such as extra-particle aerial mycelial growth. The growth matrix 3 is shown as circles. In some embodiments, the growth matrix 3 can be contained within a tray 11 with a bottom and side walls as shown. The growth matrix 3 can include a growth medium and a fungus. For example, the growth matrix 3 can comprise growth media 2, substrate 1, and colonized substrate 6, to support growth therefrom. In some embodiments, the growth matrix 3 is implemented without tray 11 (e.g., on another growth support structure, such as a porous or nonporous mycological growth web, netting, or bed-like structure). The growth matrix 3 can substantially lower moisture content relative to a liquid culture (e.g., liquid fermentation) process.

FIG. 1B illustrates an embodiment of extra-particle aerial mycelial growth 8 from the growth matrix 3 of FIG. 1A. For example, the growth can occur when the growth matrix 3 from FIG. 1A is incubated or otherwise processed within a growth environment under growth conditions suitable for the desired properties of the extra-particle aerial mycelium growth 8 in FIG. 1B.

The extra-particle aerial mycelium growth can extend away from and outward from a surface of the growth matrix to form an aerial mycelium 7 as shown. Appropriate growth conditions of the growth matrix 3 in FIG. 1A result in extra-particle aerial mycelium growth initiating across the exposed surface. Next, extra-particle aerial mycelium growth continues to expand forming a volume of extra-particle mycelial growth 8 as shown in FIG. 1B. The volume of growth can be contiguous. The extra-particle aerial mycelium growth 8 can be grown to various heights. In some embodiments, the growth is about 3-4 inches high above the growth matrix 3. It will be understood that although the extra-particle aerial mycelium growth has some amount of irregularity to its upper surface topology as shown, the drawings are not to scale, and the top surface can be relatively flat. In some embodiments, the top surface may be processed in additional steps, to improve the surface flatness, uniformity, and/or other desirable features.

FIG. 1C illustrates an embodiment in which the extra-particle aerial mycelium growth 8 and growth matrix 3 in FIG. 1B have been divided along a separation zone 9 (dot-dashed line) to form a separated aerial mycelium 12 and depleted growth matrix 4. Referring also to FIG. 1B, the separation zone 9 (dot-dashed line) can be a zone (e.g., plane) where the extra-particle aerial mycelium growth 8 can be divided and detached from the growth matrix 3. As shown in FIG. 1C, upon detachment, the extra-particle aerial mycelial growth 8 from FIG. 1B has formed a separated aerial mycelium 12, and the growth matrix 3 from FIG. 1B has formed a depleted growth matrix 4 from which at least some nutritional content has been used. The growth environment and growth conditions in FIGS. 1A-1B form an extra-particle aerial mycelium growth with sufficient structural characteristics such that the separated aerial mycelium 12 can be a self-supporting structure, and/or can form a growth grain, as discussed elsewhere herein. A separated aerial mycelium with these advantageous characteristics is not achievable through liquid culture (e.g., fermentation) mycelial processes. The separated aerial mycelium can also have a different moisture content, and/or a different density, even without further processing, than a mycelial material grown through a liquid culture process.

The separation zone 9 can be positioned, and thus the growth matrix 3 and the extra-particle aerial mycelium growth 8 can be divided, such that the depleted growth matrix 4 includes a transitional layer 14 of extra-particle aerial mycelium growth 8 remaining upon the underlying remainder portion 17 of the depleted growth matrix 4. This transitional layer can prevent any of the growth matrix material 3 from remaining on the separated aerial mycelium 12 after detachment from the extra-particle aerial mycelium growth 8 and can allow for a cleaner, sharper detachment. The inclusion of a transitional layer 14 can be beneficial, for example, in food applications, where the product resulting from the separated aerial mycelium 12 may not be allowed to include any significant amount of growth matrix 3. The separation zone 9 need not be linear as shown, although in some embodiments, it can form a plane extending along the dot-dashed lines shown and approximately perpendicular into the view as shown, to form a plane of separation. For embodiments that implement the tray 11, the division of the extra-particle aerial mycelium growth 8 from the growth matrix may result in portions 16 of the extra-particle aerial mycelium growth 8 that extend below the separation zone 9 to be divided and detached from the separated aerial mycelium 12.

FIG. 1D illustrates an embodiment of the separated aerial mycelium 12 in FIG. 1C that has been cut to form an aerial mycelium panel 13. The cut can be made transversely into the page in the orientation shown. This cut would be across the width of the separated aerial mycelium 12. The portions 15 that are shown cut away from the aerial mycelium panel 13 may be, in some embodiments, part of other aerial mycelium panels 13 that are longitudinally adjacent to panel 13. For example, embodiments without a tray 11 and which implement a mycological web may include adjacent panels 13 rather than portions 15.

The dividing and cutting processes described with reference to FIGS. 1C and 1D, respectively, can be performed with any dividing instrument and cutting instrument, respectively, suitable to divide and/or cut mycelial growth.

For example, these instruments can comprise a wire saw, a reciprocating saw (e.g., a reciprocating blade or wire), a recirculating saw (e.g., a recirculating band or wire), any of which can be configured to move in two axes—e.g., across the width of the separated aerial mycelium, and transversely, and/or a single axis “guillotine” cut (e.g., transversely), or combinations thereof. In some embodiments, a rotating cutting instrument, such as a cutting mill or “pizza cutter” configuration, can be implemented. In some embodiments, a double cake slicer blade can be implemented.

FIG. 2 illustrates an embodiment of a process 200 for making an edible mycelium-based food product, such as a mycelium-based bacon.

The process 200 begins with step 202 where one or more aerial mycelium segments are provided. The one or more aerial mycelium segments can be provided after growing a self-supporting aerial mycelium with a growth grain in the manner described above in conjunction with FIGS. 1A and 1B (and below with respect to step 302 in FIG. 3), processing the aerial mycelium into an aerial mycelium panel (or more generally, any aerial mycelium segment), as shown above in FIGS. 1C and 1D (and below with respect to step 304 in FIG. 3), and forming one or more aerial mycelium segments, as shown below with respect to step 306 in FIG. 3, or through other aerial mycelium growth and panel processes. In some embodiments, additional steps may be performed on the aerial mycelium, the aerial mycelium panel, and/or aerial mycelium segments prior to step 202, such as compressing, stretching, shaping, soaking, heating, cooling, freezing, wetting, drying, marbling, etc. For example, the aerial mycelium panel may be compressed to form a higher density material and shaped to fit into a cutting machine.

Next, the process 200 moves to step 204 where the aerial mycelium segments are processed in either a vacuum chamber, or specifically a vacuum tumbler (as a singular device), or in a particular sequence of steps in various devices to form processed aerial mycelium segments. The aerial mycelium segments and the processed aerial mycelium segments may be substantially the same shape. Processing the aerial mycelium segments in the vacuum chamber or vacuum tumbler (or among various devices) may include in a singular device or separate devices, the steps of boiling the aerial mycelium segments, flavoring the aerial mycelium segments, drying the aerial mycelium segments, and/or other processing steps. In some embodiments, processing the aerial mycelium segments in the vacuum chamber may not include altering the shape and/or dimensions of the aerial mycelium segments. For example, in some embodiments, processing the aerial mycelium segments in the vacuum chamber, and/or one, more, or all of the additional processing steps herein can be performed without a molding (e.g. vacuum molding) step, and the apparatus and systems herein can perform said steps without requiring a mold to form the desired shape of the mycelium products.

The aerial mycelium segments may be flavored with a brining fluid, marinade, seasoning, and/or rub in the vacuum chamber or vacuum tumbler, as discussed in greater detail below. In some embodiments, the one or more aerial mycelium segments may be flavored in a vacuum tumbler or device (or vacuum devices with similar functionality). The aerial mycelium segments may also be dried in the vacuum tumbler. Drying may include reducing the moisture content of the one or more aerial mycelium segments, for example, before or after the flavoring step. In some embodiments, the one or more aerial mycelium segments may be dried and/or cured in the vacuum chamber or vacuum tumbler at a particular temperature and a particular pressure. In some embodiments, curing may include dry curing, brine curing, wet curing, or acid curing. Brine curing, wet curing, and acid curing may not involve a previous injection step. Instead, the one or more aerial mycelium segments may be wet cured, brine cured, and/or acid cured after the one or more aerial mycelium segments are flavored.

In a non-limiting example of step 204, the one or more aerial mycelium segments may be processed in a vacuum chamber, such as the vacuum tumbler 400 shown and described further below with respect to FIG. 4. The aerial mycelium segments may be loaded into the vacuum tumbler 400 and boiled in an aqueous solution until the segments reach a target internal temperature. The aqueous solution may be removed from the tumbler 400, and a brining fluid may be added. The tumbler 400 may reduce the pressure in the vessel to a flavoring pressure and mix the brining fluid and the aerial mycelium segments for a time period. Next, excess brining fluid may be removed from the tumbler 400, and the tumbler 400 may reduce the pressure in the vessel to a drying pressure and bring the temperature in the vessel to a drying temperature. After a drying time has elapsed, the processed aerial mycelium segments may be removed from the tumbler 400. As discussed above, the shape of the aerial mycelium segments may be maintained during processing in the vacuum chamber, such as a vacuum tumbler.

In some embodiments, the process 200 may include more or fewer steps. In some embodiments, one or more of the steps of process 200 may be performed in a different order or simultaneously with respect to one or more of the other steps of process 200. Additionally, although the process 200 is described as processing an aerial mycelium segment, it will be understood that an aerial mycelium, an aerial mycelium panel, or any portion thereof may be used.

FIG. 3 illustrates an embodiment of a process 300 for making an edible mycelium-based food product, such as a mycelium-based bacon. The process 300 can begin with step 302 of growing an aerial mycelium. Any of the growth methods described herein, or other methods, may be employed and may be mixed and matched to achieve an aerial mycelium with a particular morphology, such as a particular density, length, etc. For example, an aerial mycelium may be grown in the manner described above in conjunction with FIGS. 1A and 1B.

The process 300 then moves to step 304 of processing the aerial mycelium into an aerial mycelium panel. Step 304 can include the process depicted in FIGS. 1C-1D, along with its alternatives or other steps suitable to form an aerial mycelium panel from an aerial mycelium. In some embodiments, step 304 may encompass additional steps described above, or other steps, such as compressing, stretching, shaping, soaking, heating, cooling, freezing, wetting, drying, marbling, etc. For example, the aerial mycelium panel may be compressed to form a higher density material and/or shaped to fit into a cutting machine.

Next, the process 300 can move to step 306 where the aerial mycelium panel is cut into one or more aerial mycelium segments. The one or more aerial mycelium segments refer to a portion of the aerial mycelium panel that is smaller than the whole aerial mycelium panel. For example, the aerial mycelium panel may be cut into one or more strips, e.g. rectangular strips. In some embodiments, one or more aerial mycelium panel(s) may be processed whole with the process 300, without being cut into smaller segments. Additionally, although the process 300 is described as processing aerial mycelium segments and aerial mycelium panels, it will be understood that an aerial mycelium, an aerial mycelium panel, or any portion thereof may be used. In some embodiments, a whole aerial mycelium or any portion thereof may be provided and made into an edible mycelium-based food through one or more of the steps of process 300.

To form one or more aerial mycelium segments, the aerial mycelium panel can be cut in any direction, such as with the growth grain or against the growth grain. As it is an object of the present disclosure to provide a food product or ingredient having the look and mouth-feel of a whole cut of animal-based meat, the mycelial growth grain may be retained, in whole or at least in part. Thus, in some aspects, step 306 may exclude cutting, shearing, grinding and/or “mincing” the aerial mycelium panel against the growth grain. As discussed above, the aerial mycelium panel may have a growth grain. However, other mycelium culture methods, such as liquid culture (e.g., liquid fermentation), do not produce a mycelium with a such growth grain.

Cutting can be achieved by a variety of means, including but not limited to cutting with a knife, a meat or deli slicer, a bacon slicer, an ultrasonic cutter, a water jet cutter, a bandsaw, and the like. For example, a horizontal bandsaw or a multiblade bread slicer may be used. In some embodiments, the particular machinery employed may be based on a desired cut size. In a non-limiting example, a horizontal band saw may be used to cut the aerial mycelium panel into 5 mm thick segments. In another non-limiting example, a multiblade bread slicer may be used to cut the panel into 9.5 mm thick segments.

According to some embodiments, the aerial mycelium panel may be partially or wholly frozen for cutting or slicing. In some embodiments, a crust of the aerial mycelium panel may be frozen. The thickness of the frozen crust may be between about 1 mm and 20 mm. In some embodiments, the thickness of the frozen crust may be between about 1 mm and 10 mm. In a non-limiting example embodiment, an aerial mycelium panel may be completely frozen and sliced by an involute rotary slicing blade. In some embodiments, the aerial mycelium panel may be frozen as the panel is sliced. For example, the face of the aerial mycelium panel to be cut or sliced can be cooled, frozen, or flash frozen as the panel is sliced. Advantageously, freezing the aerial mycelium panel prior to or during slicing allows the panel to be cut faster without damaging the panel or segments. In turn, freezing the panel during slicing produces more segments and less waste in less time. Such controlled freezing of a portion of the crust of the panel can provide slicing advantages without damaging the mycelial cell structure of the entire panel, which can occur if the entire panel is frozen. Although freezing of an aerial mycelium is described, an aerial mycelium or any portion thereof may be frozen before and/or during a cutting process.

Next, the process 300 can include one or more processing steps on the aerial mycelium segment, such as processing steps 308, 310, and 312, to form a processed aerial mycelium segment. In some embodiments, two or more of steps 308, 310, and 312 can be performed in the same type of vessel. For example, in some embodiments, two or more of steps 308, 310, and 312 can be performed on the same aerial mycelium segments within the same vessel, e.g., without removing the aerial mycelium segments from the vessel between steps. The process 300 may be implemented such that it does not include or require a molding step. This can simplify the process, the amount of processing equipment needed, increase efficiency and output, and decrease costs.

During processing in a vacuum chamber, the aerial mycelium segments may maintain their shape. In other words, the shape of the aerial mycelium segments may not substantially change. For example, a rectangular aerial mycelium segment may maintain its rectangular shape and not be processed, e.g. molded, to form an oblong aerial mycelium segment. In some embodiments, the aerial mycelium segments may maintain their shape by maintaining at least one dimension, maintaining at least two dimensions, or maintaining at least three dimensions. In some embodiments, the aerial mycelium may maintain one or more material properties during processing in a vacuum chamber. For example, a grain direction of the aerial mycelium segments may be maintained during processing in a vacuum chamber.

For example, process 300 moves to step 308 where the one or more aerial mycelium segments can be boiled. In some aspects, the boiling may modify or denature proteins within the one or more aerial mycelium segments, i.e. “cook” the segments. Boiling may also reduce moisture, disinfect, reduce, or remove native compounds and/or malodors, and/or reduce bitterness. In a non-limiting example, the one or more aerial mycelium segments can be boiled to remove undesirable compounds or other substances.

The aerial mycelium segments may be boiled in an aqueous solution. In some embodiments, the aqueous solution comprises one or more additives. In some more particular embodiments, the aqueous solution contains salt. In some embodiments, the aqueous salt solution can have a salt concentration of at most about 35% (w/w) (i.e., a saturated saline solution), or alternatively at most about 26% (w/w). In some embodiments, the salt concentration is within a range of about 0.1% (w/w) to about 35% (w/w), about 0.1% (w/w) to about 26% (w/w) , about 0.1% to about 15% (w/w), about 0.5% to about 10% (w/w), about 0.5% to about 5% (w/w) or about 1% to about 3%. In some embodiments, the salt is sodium chloride. Other additives can include but are not limited to flavorants and/or colorants. The time and/or temperature of the boiling and the concentration of the salt and any additives can be adjusted by the skilled person to achieve the desired salt content, additive content, moisture content, protein denaturization, sterility, native compound and/or malodors content or the like in the resulting boiled composition or final product.

The one or more aerial mycelium segments may be boiled for a set time or until the segments reach a target internal temperature. For example, the one or more segments may be boiled until the internal temperature of the segments reaches about 145° F., 150° F., 155° F., 160° F., or 165° F. (about 63° C., 65° C., 68° C., 71° C., 74° C., respectively). In some embodiments, the aqueous solution may be maintained at a target temperature, and the one or more aerial mycelium segments may be submerged in the aqueous solution for a set amount of time such as 10 minutes, 20 minutes, 30 minutes, 45 minutes, 1 hour, etc. In some embodiments, boiling the aerial mycelium segments may comprise exposing the segments to steam for a set time or until the segments reach a target internal temperature.

Boiling may be achieved by a variety of means and vessels. For example, the one or more aerial mycelium segments may be boiled in a kettle or a vacuum chamber, such as a vacuum tumbler. For example, the one or more aerial mycelium segments may be boiled directly in a vacuum tumbler, such as a modified version of the Provisur Lutetia Tumbler manufactured by Provisur Technologies described in U.S. Pat. No. 10,743,555, the entirety of which is incorporated herein by reference thereto, except where inconsistent with the disclosure herein. For example, the Type 0 Lutetia Tumbler, the Type 1 Lutetia Tumbler, the

Type 2 Lutetia Tumbler, the Type 3 Lutetia Tumbler, the Type 4 Lutetia Tumbler, the Type 40 Lutetia Tumbler, the Type 400 Lutetia Tumbler, the Type 5 Lutetia Tumbler, the Type 6 Lutetia Tumbler, or the Type 7 Lutetia Tumbler may be employed. In another example, an LT-30 tumbler from Lance Industries may be employed. The vacuum tumbler may inject steam into the vessel and boil the one or more aerial mycelium segments until the internal temperature of the segments reaches a desired amount, such as in a range of about 135° F. to about 212° F., in a range of about 165° F.-190° F., and/or about 165° F. (about 74° C.). In some embodiments, water or an aqueous solution may be added to the vacuum tumbler along with the one or more aerial mycelium segments and heated until it boils. In some embodiments, the use of a vacuum chamber, such as a vacuum tumbler, can allow the boiling step to be implemented using steam at a higher temperature (e.g., thus faster) than conventional processes.

In some embodiments, the one or more aerial mycelium segments may be boiled in a vacuum chamber. In some embodiments, the aerial mycelium segments may be boiled first before being placed in a vacuum chamber for additional processing. The aerial mycelium segments may be placed in a low-pressure environment for boiling. A low-pressure environment is an environment with an air pressure less than atmospheric pressure (1013.25 millibars) up to and including a vacuum (0 millibars). For example, the one or more aerial mycelium segments may be boiled in a vacuum tumbler at a pressure between about 200 and about 600 millibars (mbar), such as about 350 mbar.

The process 300 moves to step 310 where the one or more aerial mycelium segments are flavored. Flavoring the one or more aerial mycelium segments may include marinating the segments, brining the segments, applying a rub to the segments, soaking the segments, seasoning the segments, and/or other flavoring steps. The flavoring step 310 can comprise a dry, wet, or a combined dry and wet flavoring process. In some embodiments, step 310, or a step similar to 310, may be implemented to impart a color to the one or more aerial mycelium segments.

Flavoring the aerial mycelium segments can include contacting the one or more aerial mycelium segments with a flavoring component. A flavoring component may be a mixture or solution that includes one or more of: water, oil(s), fat(s), salt(s), acid(s), spice(s), sweetener(s), flavorant(s), colorant(s), vitamin(s), mineral(s), amino acid(s), and/or other additive(s), such as other additive(s) described herein. For example, the flavoring component may be a brining fluid or a marinade. In some embodiments, a flavoring component can be an aqueous solution containing salt. The aqueous salt solution can have a salt concentration of at most about 35% (w/w) (i.e., a saturated saline solution), or alternatively at most about 26% (w/w). In some embodiments, the salt concentration is within a range of about 0.1% (w/w) to about 35% (w/w), about 0.1% (w/w) to about 26% (w/w) about 0.1% to about 15% (w/w), about 0.5% to about 10% (w/w), about 0.5% to about 5% (w/w) or about 1% to about 3%. In some embodiments, the salt is sodium chloride. In some embodiments, the flavoring component does not contain a salt. In some embodiments, the brining fluid is a non-aqueous solution.

In another example, the flavoring component may include thiamine. Thiamine may be degraded into a plurality of compounds (e.g., thiols, sulfides, disulfides, etc.) which may react with one or more compounds in the aerial mycelium (e.g., sugars and/or amino acids) to produce molecules associated with meaty flavors and/or meaty aromas. The addition of thiamine facilitates Maillard reactions which produce molecules associated with meaty flavors and/or meaty aromas such as thiazoles, furans, pyrazines, oxazoles, aldehydes, ketones, and/or sulfur compounds (e.g., thiophenes such as 2-formyl-5-methylthiophene). During Strecker degradation, amino acids are decarboxylated and deaminated to produce aldehydes, carbon dioxide, and amines. The aldehydes formed in this process contribute significantly to the flavor of cooked foods. Thiamine may react further with these aldehydes to produce molecules associated with meaty flavors and/or meaty aroma according to some embodiments.

Other compounds may be added to the flavoring component to modify the types of molecules produced during and/or through the Maillard reactions. More specifically, the types of aldehydes formed during the Strecker degradation substep of the Maillard reaction depend, at least in part, on what amine-containing molecules and saccharides are present Accordingly, one or more compounds may be added to influence the Strecker aldehydes that can be produced. In some embodiments, the one or more compounds may be selected based on the chemical composition of the one or more aerial mycelium segments, e.g. the saccharides and amino acids present in the segment(s). The Maillard reaction(s) described above may take place any time after thiamine is added to the one or more aerial mycelium segments. For example, the reactions may occur during a pasteurization step as described below.

The amount of thiamine added may be based on an amount and/or a chemical composition of the one or more aerial mycelium segments. In some embodiments, the amount of thiamine may be based on a weight of the aerial mycelium segments. For example, thiamine may be added at a 1:1,000 (w/w) ratio to the aerial mycelium, e.g. 1 gram of thiamine for each kilogram (1,000 grams) of aerial mycelium. In some embodiments, thiamine may be added based on a chemical composition of the one or more aerial mycelium segments. Thiamine may be added based on a sugar composition of the aerial mycelium segments according to some embodiments. Thiamine may be added at a ratio between about 1:1 (w/w) and about 1:5 (w/w) of thiamine to sugar content of the aerial mycelium. For example, 1 gram of thiamine may be added for every 5 grams of sugar in the aerial mycelium segments. In some embodiments, thiamine may be added at an amount sufficient to produce a target ratio of thiamine to sugar content in the processed aerial mycelium segments. The target thiamine to sugar content of the processed aerial mycelium segments may be a ratio between about 1:1 (w/w) and about 1:5 (w/w) of thiamine to sugar content of the processed aerial mycelium. In some embodiments, sugar may also be added to the flavoring component in addition to the sugar of the aerial mycelium segments. The amount of added sugar and/or thiamine may be sufficient to produce a thiamine to sugar content of the processed aerial mycelium segments between about 1:1 (w/w) and about 1:5 (w/w). In some embodiments, the thiamine-containing flavoring component and the aerial mycelium segment(s) may be heated to a flavoring temperature at a flavoring pressure for a flavoring time. The flavoring temperature may be about 100° C. The flavoring pressure may be between about 0 mbar and about 1,000 mbar. For example, the flavoring pressure may be between about 33 mbar and 45 mbar. The flavoring time may be between about 10 minutes and about 60 minutes. For example, the flavoring time may be about 20 minutes.

Advantageously, processing the one or more aerial mycelium segment(s) and a thiamine-containing flavoring component in the vacuum chamber may provide an enhanced flavor-profile for the aerial mycelium segment(s). The chemical composition of the aerial mycelium segments, which may include various saccharides, amino acids, and a moisture content, in combination with the thiamine may produce a unique, robust flavor profile. For example, the amino acids present in the aerial mycelium segments may enhance the production of and/or presence of umami flavors. Further, the lack of oxygen in the vacuum chamber may influences the types of reactions that may take place, favoring compounds which do not require oxidative conditions to form. More specifically, processing the one or more aerial mycelium segment(s) and a thiamine-containing flavoring component in the vacuum chamber may enhance the production of thiazoles, oxzoles, furans, and pyrazines as well as limiting the amount of aldehydes and ketones.

The amount of flavoring component added to the one or more aerial mycelium segments may be based on one or more of: a weight of the segments, a volume of the segments, and a surface area of the segments. In some embodiments, the flavoring component may be added to the aerial mycelium segments at a 0.25:1, 0.5:1, 1:1 , 2:1, 3:1, 4:1, or 5:1 ratio of flavoring component volume to aerial mycelium segment volume. In a non-limiting example embodiment, 3 liters of flavoring component may be added for every 1 liter of aerial mycelium segments. In some embodiments, the flavoring component may be added to the aerial mycelium segments at a 0.25:1, 0.5:1, 1:1 , 2:1, 3:1, 4:1, or 5:1 ratio of flavoring component mass to aerial mycelium segment mass. For example, the flavoring component may be added at a 1:1 (w/w) ratio of flavoring component to aerial mycelium segment(s).

In some embodiments, step 310 may also comprise imparting a mechanical action such as stirring, tumbling, shaking, agitating, or other actions, directly or indirectly onto the one or more aerial mycelium segments and/or the flavoring component. This mechanical action step can be implemented before, during or after step 310, and/or before, during or after any other processing steps, such as steps 308 or 312.

In some embodiments, the one or more aerial mycelium segments may be flavored in a low-pressure environment. In some more particular embodiments, the one or more aerial mycelium segments may be flavored at a pressure less than 1013.25 mbar (atmospheric pressure), at a pressure less than or equal to about 900 mbar, less than or equal to about 800 mbar, less than or equal to about 700 mbar, less than or equal to about 600 mbar, less than or equal to about 500 mbar, less than or equal to about 400 mbar, less than or equal to about 300 mbar, less than or equal to about 200 mbar, less than or equal to about 100 mbar, greater than zero mbar, or any range therebetween. Similar ranges may in some embodiments be implemented for other processing steps.

In some embodiments, the aerial mycelium segments may be flavored at a particular temperature. The one or more aerial mycelium segments may be flavored between 0° F. and 200° F. (−18° C. and 93° C., respectively). For example, the aerial mycelium segments may be flavored at about or below 40° F., an ambient temperature such as 70° F., or 160° F. (4° C., 20° C., or 71° C., respectively).

In some embodiments, the aerial mycelium segments may be flavored in the same type of vessel that the segments were boiled in, as described with respect to step 308. For example, the vessel may be configured to both boil and flavor the aerial mycelium segments. For example, as discussed above, the one or more aerial mycelium segments may be boiled in the vessel of a vacuum tumbler. After boiling, the aqueous solution may be removed and an amount of flavoring component may be added to the tumbler based on the volume, surface area, and/or weight of the one or more aerial mycelium segments. The pressure in the vessel can be reduced and begin tumbling the aerial mycelium segment(s) and the flavoring component(s) together for an effective amount of time.

In some embodiments, flavoring the one or more segments of the aerial mycelium may include contacting the one or more aerial mycelium segments with a first flavoring component at a first flavoring pressure and a first flavoring temperature and contacting the one or more aerial mycelium segments with a second flavoring component at a second flavoring pressure and a second flavoring temperature. The first flavoring pressure and the first flavoring temperature may be based on one or more properties of the first flavoring component such as the chemical composition of the first flavoring component. Similarly, the second flavoring pressure and the second flavoring temperature may be based on one or more properties of the second flavoring component. In some embodiments, an amount of the first flavoring component added to the vacuum chamber and/or a ratio of the first flavoring component to aerial mycelium (w/w or v/v) may be based on the one or more properties of the first flavoring component and/or one or more properties of the aerial mycelium (size, density, etc.). Similarly, the amount of the second flavoring component added to the vacuum chamber and/or a ratio of the second flavoring component to aerial mycelium (w/w or v/v) may be based on the one or more properties of the second flavoring component and/or one or more properties of the aerial mycelium (size, density, etc.).

Advantageously, flavoring the one or more segments of aerial mycelium in a low-pressure environment can allow the flavoring component to be more readily absorbed by or placed within or adhered to the segment(s). Aerial mycelium is a hydrophobic material with low water uptake, which prevents or resists absorption of aqueous marinades and flavor additives, unlike many animal-based meats and other food products. However, aerial mycelium is also compressible. By contacting the one or more aerial mycelium segments having a growth grain as described above with a flavoring component in a low-pressure environment, the aerial mycelium can act like a sponge, soaking up and capturing more of the flavoring component(s) and associated flavors than other flavoring methods, like injection (and without the possible negative structural consequences, like semi-permanently or permanently formed pockets in the biomaterial). Flavoring the aerial mycelium in a low pressure environment can provide better flavor absorption than other flavoring methods, such as flavor injection, or by pressurizing flavorants across an outer surface of the aerial mycelium. In turn, a more flavorful mycelium-based food product can be produced that more closely mimics the rich flavors of other foods. For example, using the methods described herein, a more flavorful mycelium-based bacon can be produced that has similar flavors and texture as animal-based bacon. Unlike traditional animal-based bacon, the mycelium-based bacon can be produced with a smaller carbon footprint, and other benefits.

Next, the process 300 moves to step 312 where the aerial mycelium segments are dried. Step 312 can include heating or cooling the segments. The heating can be performed by any variety of means, including a conventional oven, a convection oven, a microwave, a dehydrator or a freeze dryer or the like. The drying time and means can be adjusted by the skilled person to achieve the desired moisture content of the resulting dried composition or final product.

In some embodiments, the drying step 312 may comprise curing the aerial mycelium strips/segments. As described above, curing may include dry curing, wet curing, brine curing, and/or acid curing. The one or more aerial mycelium segments may be dried and/or cured in a low-pressure environment. In some embodiments, the aerial mycelium segments may be dried or cured in a vacuum tumbler specifically. Alternatively, the segments may be handled and processed in a series of steps/devices that function like a vacuum tumbler. In some more particular embodiments, the vacuum tumbler may be configured to boil, flavor, and dry the one or more aerial mycelium segments. In a non-limiting example, the vacuum tumbler may control the pressure within the vessel to a pressure between about 3 and about 50 mbar, and in some embodiments, between about 5 and about 40 mbar for the drying step 312.

The one or more aerial mycelium segments may be dried at a desired drying temperature, for a desired drying time. In some embodiments, the aerial mycelium segments may be dried at about 250° F. (121° C.) for 15 minutes. However, the drying time and temperature can be adjusted by the skilled person to achieve the desired moisture content and flavor of the resulting aerial mycelium segments. In some embodiments, the drying time may be determined based on the volume and/or dimensions of the aerial mycelium segments. For example, if the aerial mycelium segments are less than 25 mm thick, the drying time can be from about 20 minutes to about 60 minutes. If the aerial mycelium segments are between 25 mm and 150 mm thick, the drying time can be from about 8 hours to about 12 hours. For comparison, the drying time for aerial mycelium segments less than 25 mm can be from about 2 hours to about 4 hours at atmospheric pressure, and the drying time for aerial mycelium segments between 25 mm-150 mm may be from about 12 hours to about 24 hours at atmospheric pressure. In some embodiments, the drying temperature can be from about or below 40° F. to about 70° F. (about 4° C. to about 20° C.). As discussed above, the aerial mycelium segment(s) may maintain their shape during processing. Thus, the shape of the aerial mycelium segment(s) may be substantially the same after a drying step as when the aerial mycelium segment(s) were produced. In some embodiments, the one or more aerial mycelium segments may maintain at least one dimension, maintain at least two dimensions, or maintain at least three dimensions across and/or during one or more processing steps.

Advantageously, unlike curing animal-based meat which must be done at relatively low temperatures to prevent microbial growth, the aerial mycelium segments may be cured at higher temperatures, which allows the aerial mycelium segments to dry faster and capture more flavor. For example, the aerial mycelium segments may be cured at non-refrigerated temperatures, which provides additional energy savings along with an improved mycelium-based food product. Additionally, curing at a higher temperature allows for greater capture of other additives such as fat that can be added to the aerial mycelium segments during or after curing. In turn, a more delectable mycelium-based food product can be created in an exponentially shorter period of time. It will be understood that these advantages discussed in the context of curing can be realized more generally in the context of the drying processes described herein.

In some embodiments, method 300 can include a step of introducing air, (e.g., injecting air) into the vacuum tumbler. The introducing air step can be implemented, for example, before, during, or after one or more of the processing steps 308-312, or other steps. In some embodiments, air can be introduced while the aerial mycelium segments are being dried (e.g., cured). In turn, the drying rate of the one or more aerial mycelium segments may be increased. More specifically, introduction of air may remove liquid water quicker and increase surface drying on the one or more aerial mycelium segments. The air may be compressed air, e.g., to a desired pressure. The air may also be preconditioned. For example, in some embodiments, the air may be a particular temperature as or before it is introduced into the vessel. For example, the air may be room temperature (approximately 68° F.), above 70° F., above 80° F., above 90° F., above 100° F., above 110° F., or above 120° F. (i.e., approximately 20° C., above 21° C., above 27° C., above 32° C., above 38° C., above 43° C., or above 49° C.). In some embodiments, the air may be preconditioned to a particular humidity. For example, the air may have a relative humidity of 50% or less, 40% or less, 40% or less, 30% or less, 20% or less, 10% or less, or 5% or less. In some embodiments, the air may comprise an aerosol. In some embodiments, one or more additives may be added to the one or more aerial mycelium segments immediately prior to, during, and/or after step 312. The introduction of air generally, and in some embodiments, the temperature, pressure, air flow rate, humidity, and/or other preconditioning of the air, can prevent moisture build up and temperature drops caused by the vacuum in the vessel. In some embodiments, the conditioning of the air can be controlled to maintain an approximately constant temperature during the processing step(s).

In some embodiments, the air may be introduced at the end of a drying time. In some embodiments, the air may be introduced to the chamber or tumbler vessel in a cycle. For example, the vacuum tumbler may reduce the pressure in the vessel to a first pressure, and after a first time period, compressed air may be introduced to the vessel, increasing the pressure in the vessel to a second pressure for a second time period. The vacuum tumbler may then reduce the pressure to the first pressure again for a third time period. Air may then be introduced to the vessel again and raise the pressure in the vessel back to the second pressure. In some embodiments, the air may be continuously cycled through the vacuum tumbler such that the air remains the same temperature and relative humidity in the vacuum tumbler over a period of time, or at various points in a processing cycle.

Following step 312, the dried aerial mycelium segments may be referred to as processed aerial mycelium segments. These terms are not meant to be limiting but are intended to reflect that the one or more aerial mycelium segments have been further processed after the initial mycelium growth and panel formation, and in some embodiments, segmenting (aka further sizing). In some embodiments, the processed aerial mycelium segments can refer to a bulk mycelium-based food product, such that the segments are edible.

Next, the process 300 moves to step 314 where the bulk mycelium-based food product is portioned. Portioning the bulk mycelium-based food product may comprise dividing the bulk food product into consumer portions based on a weight or volume. For example, the bulk mycelium-based food product may be divided into a plurality of 0.5-pound (lb) portions, 1 lb portions, 1.5 lb portions, or 2 lb portions. In some embodiments, the bulk mycelium-based food product may be portion based on a number of aerial mycelium segments. For example, where the aerial mycelium segments are bacon-sized segments, the bulk mycelium-based food product may be divided into portions of 5 segments, 10 segments, 12 segments, 15 segments, 20 segments, 24 segments, etc. In some embodiments, the bulk mycelium-based food product may be processed further prior to portioning. The bulk mycelium-based food product may be cut, coated, minced, seared, simmered, boiled, torched, heated, cooled, frozen, etc. For example, the bulk mycelium-based food product may be cut (or segmented) into a smaller size prior to or during portioning.

Lastly, the process 300 moves to step 316 where the portioned bulk mycelium-based food product is packaged. For example, each portion of the bulk mycelium-based food product may be vacuum sealed in individual portion-sized packaging, or multiple portion-sized packaging. In some embodiments, the portioned bulk mycelium-based food product may be pasteurized prior to packaging. In some embodiments, the portioned bulk mycelium-based food product may be suspended in an aqueous solution within the vacuum-sealed individual packaging. In some embodiments, the mycelium-based food product may be pasteurized in the vacuum-sealed individual packaging. Pasteurization may include heating the mycelium-based food product in the vacuum-sealed individual packaging at a pasteurization temperature for a pasteurization time. For example, the mycelium-based food product may be boiled in the vacuum-sealed individual packaging. Heating the mycelium-based food product to the pasteurization temperature may facilitate chemical reactions in the mycelium-based food product. For example, heating the mycelium-based food product may facilitate Maillard reactions, e.g. Maillard reactions that generate “meaty flavor” or “meaty aroma” compounds through degradation of thiamine as described above. The pasteurization temperature and/or pasteurization time may be based on one or more properties of the individual packaging, e.g. a thickness of the packaging, and/or one or more material properties of the mycelium-based food product, e.g. one or more dimensions of the processed aerial mycelium segments. For example, the pasteurization time may be about 20 minutes. The pasteurization temperature may be about 212° F. [0176] In some embodiments, the process 300 may include more or fewer steps. In some embodiments, one or more of the steps of process 300 may be performed in a different order or simultaneously with respect to one or more of the other steps of process 300. Additionally, although the process 300 is described as processing an aerial mycelium segment, an aerial mycelium, or any portion thereof, may be used.

The process 300 (and more generally, process 200) advantageously provides a more flavorful, succulent mycelium-based food product than was previously available. Because the process 300 allows an aerial mycelium to better absorb or retain and capture a flavoring component faster, the process 300 takes substantially (e.g., exponentially) less time such that mycelium-based products can be produced in a matter of hours (or potentially less) rather than days, weeks or even months, compared to many animal-based meat or other products. Furthermore, the end product is a more delectable mycelium-based food product that more precisely mimics other animal-based foods, such as animal-based bacon.

### Vacuum Tumbler

FIG. 4 illustrates an example embodiment of a vacuum tumbler 400 that may be configured to process one or more aerial mycelium segments. For example, the vacuum tumbler 400 can be implemented to form one or more processed aerial mycelium segments, as described herein with respect to FIGS. 2 and 3, including the boiling, flavoring, and drying steps, or through other processes or steps (such as curing).

The vacuum tumbler 400 may comprise a body 402 that forms a vessel configured to receive one or more segments of aerial mycelium 404. The body 402 may be cylindrical and sized to receive a particular volume of aerial mycelium for processing. In some embodiments, the body may be an insulated structure. For example, the body may comprise a double walled vessel that includes an interior wall and an exterior wall that are separated by a vacuum space. In some embodiments, the body 402 may be manufactured from stainless steel. At least some portions of the body 402 may be coated to reduce sticking of aerial mycelium to the interior surface during processing and/or to prevent microbial growth. In some embodiments, the interior surface of the body may comprise an antimicrobial microstructure or nanostructure, and/or other materials or configurations. At a proximal end of the body 402, a lid 406 may be disposed. Although a hinged lid 406 is depicted in FIG. 4, the depiction of the lid 406 is not intended to be limiting. For example, the lid 406 may be a circular lid with a locking wheel mechanism. The lid 406 can comprise any configuration suitable to allow introduction of aerial mycelium into the body 402 and removal of processed aerial mycelium from the body 402. The lid 406 and the body 402 can be configured of and/or comprise materials with sufficient strength and sealing capabilities to allow for desired pressure, temperature, and other aerial mycelium processing parameters described herein, or other parameters.

The vacuum tumbler 400 may further comprise an actuator 408, a vacuum pump 410, a fluid pump 412, a temperature control element 414, and one or more sensors 416.

Actuator 408 can comprise a component or system that is configured to cause relative motion (e.g., rotational, linear, pivoting, vibrational, or ultrasonically generated motion) between two or more components (e.g., a motion between the body 402 and a rotational axis). Actuator 408 can comprise one or more of, or a combination of, e.g., a hub, bearing, hinge, pin, ball and pinion, axle, rotational joint, clutch, disc, gear, belt, motor, linear slide, linear actuator, track, groove, slot, cam, vibrational table, etc. It will be understood that an actuator is not necessarily tied to an electronic, motorized, or otherwise automatic system, and that embodiments of the actuator(s) described herein can be configured to be moved manually, semi-automatically, and/or automatically. For example, the actuator 408 may be mechanically connected to the body 402 and configured to rotate the body 402, thereby mixing the aerial mycelium segments 404 with another component such as an aqueous solution for boiling or a flavoring component. In some embodiments, the actuator 408 may be configured to mix the one or more aerial mycelium segments 404 by shaking the body 402, rocking the body 402, agitating the body 402, etc. It will be understood that these mechanical actions can be imparted upon the aerial mycelium segment(s) and/or flavor component(s) (e.g., within the vacuum tumbler 400), similar to those mechanical actions described above with respect to FIG. 2. Additionally, these mechanical actions can be implemented before, during, or after one or more of the various processing steps described herein, for example, with respect to FIGS. 2 and 3.

The vacuum pump 410 may be in fluid communication with the body 402. For example, the vacuum pump 410 may be connected to the body 402 via piping. The vacuum pump 410 may remove air out of the body 402 to decrease the pressure therein (e.g., to create a low-pressure environment). The vacuum pump 410 may introduce (e.g., pump) air into the body 402. As such, the vacuum pump 410 can generally be configured to maintain a particular pressure or pressure profile within the body 402. For example, the vacuum pump 410 can be configured to cycle the pressure within the body 402 between two or more pressures.

In some embodiments, the vacuum tumbler 400 may include a fluid pump 412 that is in communication with the interior of the body 402. The fluid pump 412 may be any configuration suitable to introduce or remove fluids and/or other dry, wet, vaporized, and/or gaseous process components into or out of the body 402 for various processes. For example, the fluid pump 412 may be configured to introduce an aqueous solution and/or steam into the body 402 for boiling. The fluid pump 412 may be configured to remove aqueous solution and/or steam from the body 402, e.g., after boiling is completed, and/or during a drying step. The fluid pump 412 may be configured to introduce a flavoring component into the body 402. The fluid pump 412 can comprise an active component, such as a pump, hopper/screw feeder (e.g., with a motor) and/or can comprise a passive component, such as a gravity-fed drain or feeder, or other active or passive components. In some embodiments, the vacuum tumbler 400 may include a separate fluid pump 412 for each component that may be introduced into or removed from the interior of the body 402.

In some embodiments, the vacuum tumbler 400 may include a temperature control element 414 that is electrically connected to the body. The temperature control element 414 may be configured to control the temperature within the body 402 by heating or cooling the body 402 and the contents of the body 402, and thus control the temperature of an aerial mycelium segment within the body 402. The temperature control element 414 may comprise a heating element, a compressor, a blower and/or other suitable components configured to affect temperature of the body 402. The temperature control element 414 may heat or cool the body 402 directly and/or indirectly by heating or cooling the air or liquid inside the body 402. The temperature control element 414 may maintain a particular temperature throughout a step, or it may cycle the temperature as directed.

The vacuum tumbler may also include one or more sensors 416. The sensor(s) 416 can be configured to detect one or more environmental conditions related to the processing of the aerial mycelium for example, within the body 402. The sensor(s) 416 can be configured to generate a first signal indicative of the environmental condition, which is then communicated to a processor configured to either increase, decrease, or maintain that environmental condition, for example, within the body 402. The one or more sensors 416 may include a temperature sensor, a pressure sensor, a humidity sensor, a weight sensor, a volatile compound sensor, and/or other sensors. For example, in some embodiments, the humidity sensor may detect a temperature within the body 402 and generate a first signal indicative of the temperature to a processor.

In some embodiments, the vacuum tumbler 400 may include one or more processors 418. The one or more processors 418 may be one or more processors electronically connected to a memory, the actuator 408, the vacuum pump 410, the fluid pump 412, the temperature control element 414, and the one or more sensors 416. The one or more processors 418 may receive a signal (e.g., data) from the one or more sensors 416 regarding the environmental conditions in the body 402. Based on the measurements from the one or more sensors 416, the one or more processors 418 may activate, deactivate, or instruct the actuator 408, the vacuum pump 410, the fluid pump 412, the temperature control element 414 and/or other components to change and thus control an environmental condition within the body 402. For example, the one or more sensors 416 may detect an unexpected pressure increase in the body 402 during a processing step. The one or more processors 418 may receive the pressure reading from the one or more sensors 416 and instruct the vacuum pump 410 to decrease the pressure until the one or more sensors 416 detect that the pressure has returned to a desired value. In some embodiments, the one or more processors 418 may automate the processes 200 and 300, or portions thereof, based on signal(s) (e.g., data) received from the one or more sensors 416. The processor(s) 418, sensor(s) 416, the temperature control element 414 and/or fluid pump 412 may be configured to maintain an approximately constant temperature within the vacuum tumbler 400.

### Scope of Disclosure

The following non-exhaustive list provides fungal genus and species which may be implemented, if not otherwise inconsistent with the present disclosure.

The fungus may be a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces or Wolfiporia.

In some further embodiments, the fungus is a species of the genus Pleurotus. In some more particular embodiments, the fungus is Pleurotus albidus, Pleurotus citrinopilleatus, Pleurotus columbinus, Pleurotus cornucopiae, Pleurotus dryinus, Pleurotus djamor, Pleurotus eryngii, Pleurotus floridanus, Pleurotus nebrodensis, Pleurotus ostreatus, Pleurotus populinus, Pleurotus pulmonarius, Pleurotus sajor-caju, Pleurotus salmoneo-stramineus, Pleurotus salmonicolor or Pleurotus tuber-regium.

The various illustrative logics, logical blocks, modules, circuits and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general-purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium. The steps of a method or algorithm disclosed herein may be implemented in a processor-executable software module which may reside on a tangible, non-transitory computer-readable medium. Computer-readable media includes both computer storage media and communication media including any medium that can be enabled to transfer a computer program from one place to another. A storage media may be any available media that may be accessed by a computer.

A software module may reside in random access memory (RAM), flash memory, read only memory (ROM), electrically programmable ROM (EPROM), electrically erasable programmable ROM (EEPROM), registers, hard disk, a removable disk, a CD ROM, or any other form of storage medium known in the art. A storage medium is coupled to the processor such that the processor can read information from, and write information to, the storage medium. In the alternative, the storage medium may be integral to the processor. Disk and disc, as used herein, includes compact disc (CD), laser disc, optical disc, digital versatile disc (DVD), floppy disk and blue ray disc where disks usually reproduce data magnetically, while discs reproduce data optically with lasers. Combinations of the above should also be included within the scope of computer readable media. The processor and the storage medium may reside in an ASIC. The ASIC may reside in a user terminal. In the alternative, the processor and the storage medium may reside as discrete components in a user terminal.

Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein. Additionally, a person having ordinary skill in the art will readily appreciate, the terms “upper” and “lower” are sometimes used for ease of describing the figures and indicate relative positions corresponding to the orientation of the figure on a properly oriented page, and may not reflect the proper orientation of a feature as implemented.

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

The word “exemplary” is used herein to mean “serving as an example, instance, or illustration.” Any aspect or embodiment described herein as “exemplary” is not necessarily to be construed as preferred or advantageous over other aspects or embodiments. Various aspects of the novel systems, apparatuses, and methods are described more fully hereinafter with reference to the accompanying drawings. This disclosure may however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of, or combined with, any other aspect described. For example, an apparatus may be implemented, or a method may be practiced using any number of the aspects set forth herein. In addition, the scope of the disclosure is intended to cover such an apparatus or method which is practiced using other structure, functionality, or structure and functionality in addition to or other than the various aspects of the disclosures set forth herein. It should be understood that any aspect disclosed herein may be embodied by one or more elements of a claim.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components, elements, and systems can generally be integrated together in a single device or system or in multiple devices or systems.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z. Thus, as used herein, a phrase referring to “at least one of X, Y, and Z” is intended to cover: X, Y, Z, X and Y, X and Z, Y and Z, and X, Y and Z.

The headings provided herein, if any, are for convenience only and do not necessarily affect the scope or meaning of the devices and methods disclosed herein.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method for producing a mycelium-based food product comprising:
providing one or more aerial mycelium segments; and
processing the one or more aerial mycelium segments in a vacuum tumbler to form one or more corresponding processed aerial mycelium segments.

2. The method of claim 1, wherein processing the one or more aerial mycelium segments comprises at least one of:
boiling the one or more aerial mycelium segments;
flavoring the one or more aerial mycelium segments; and
drying the one or more aerial mycelium segments.

3. The method of claim 2, wherein flavoring the one or more aerial mycelium segments comprises contacting the one or more aerial mycelium segments with one or more flavoring components in the vacuum tumbler at a flavoring pressure.

4. The method of claim 3, wherein the one or more flavoring components comprise one or more of: a brining fluid and a marinating fluid.

5. The method of claim 3, wherein the one or more flavoring components comprise thiamine.

6. The method of claim 5, wherein the thiamine is added based on a weight of the one or more aerial mycelium segments.

7. The method of claim 6, wherein the thiamine is added in an amount equal to about 1/1,000 of the weight to the one or more aerial mycelium segments.

8. The method of claim 5, wherein the thiamine is added based on a sugar content of the one or more aerial mycelium segments.

9. The method of claim 8, wherein the thiamine is added in an amount between about ⅕^{th} of a weight of the sugar in the one or more aerial mycelium segments to an amount equal to the weight of the sugar in the one or more aerial mycelium segments.

10. The method of claim 3, wherein contacting the one or more aerial mycelium segments with the one or more flavoring components in the vacuum tumbler comprises:
contacting the one or more aerial mycelium segments with a first flavoring component at the flavoring pressure; and
contacting the one or more aerial mycelium segments with a second flavoring component at the flavoring pressure.

11. The method of claim 3, wherein a volume of the one or more flavoring components is proportional to a volume of the one or more aerial mycelium segments.

12. The method of claim 11, wherein the volume of the one or more flavoring components is equal to between two to four times the volume of the one or more aerial mycelium segments.

13. The method of claim 3, wherein the flavoring pressure is between about 0 mbar and about 1013.25 mbar.

14. The method of claim 13, wherein the flavoring pressure is between about 0 mbar and about 100 mbar.

15. The method of claim 14, wherein the flavoring pressure is between about 30 mbar and about 45 mbar.

16. The method of claim 13, wherein the flavoring pressure is between about 26 mmHg to about 33 mmHg.

17. The method of claim 3, wherein the one or more aerial mycelium segments are contacted with the one or more flavoring components at a target temperature, wherein the target temperature is at or below 40° F.

18. The method of claim 3, wherein flavoring the one or more aerial mycelium segments further comprises mixing the one or more aerial mycelium segments and the one or more flavoring components.

19. The method of claim 2, wherein boiling comprises boiling the one or more aerial mycelium segments in an aqueous solution for a boiling time.

20. The method of claim 19, wherein the boiling time is about 20 minutes.

21. The method of claim 2, wherein boiling comprises boiling the one or more aerial mycelium segments in an aqueous solution until the one or more aerial mycelium segments reach a target internal temperature.

22. The method of claim 21, wherein the target internal temperature is between about 135° F. and about 212° F.

23. The method of claim 22, wherein the target internal temperature is between about 165° F. and about 190° F.

24. The method of claim 23, wherein the target internal temperature is about 165° F.

25. The method of claim 2, wherein drying the one or more aerial mycelium segments comprises curing the one or more aerial mycelium segments in the vacuum tumbler at a drying pressure and a drying temperature for a drying time.

26. The method of claim 25, wherein the drying pressure is between about 3 and about 50 mbar.

27. The method of claim 25, wherein the drying temperature is between about 40° F. and about 70° F.

28. The method of claim 25, wherein the drying time is between about 15 minutes and 120 minutes.

29. The method of claim 25, wherein curing the one or more aerial mycelium further comprises introducing air into the vacuum tumbler.

30. The method of claim 29, wherein the air is ambient air.

31. The method of claim 29, wherein the air is a preconditioned air with a target humidity between about 0% and 30% relative humidity.

32. The method of claim 29 further comprising removing the air from the vacuum tumbler to return the vacuum tumbler to a flavoring pressure.

33. The method of claim 2, wherein flavoring comprises fatting the one or more aerial mycelium segments with a fat, the fat selected from a group consisting of: an almond oil, an animal fat, an avocado oil, a butter, a canola oil , a coconut oil, a corn oil, a grapeseed oil, a hempseed oil, a lard, a mustard oil, an olive oil, a palm oil, a peanut oil, a rice bran oil, a safflower oil, a soybean oil, a sunflower seed oil, a vegetable oil, a vegetable shortening, or a combination thereof.

34. The method of claim 1, further comprising fatting the one or more processed aerial mycelium segments with a fat, in one embodiment, a fat selected from a group consisting of: an almond oil, an animal fat, an avocado oil, a butter, a canola oil , a coconut oil, a corn oil, a grapeseed oil, a hempseed oil, a lard, a mustard oil, an olive oil, a palm oil, a peanut oil, a rice bran oil, a safflower oil, a soybean oil, a sunflower seed oil, a vegetable oil, a vegetable shortening, or a combination thereof.

35. A method for producing a mycelium-based food product comprising:
providing one or more aerial mycelium segments;
flavoring the one or more aerial mycelium segments in a vacuum chamber at a flavoring pressure; and
curing the one or more aerial mycelium in the vacuum chamber at a drying pressure and a drying temperature for a drying time to form one or more corresponding processed aerial mycelium segments.

36. A system for producing a mycelium-based food product comprising:
one or more aerial mycelium segments; and
a vacuum tumbler comprising:
a body containing the one or more aerial mycelium segments; and
a processor configured to form one or more corresponding processed aerial mycelium segments from the one or more aerial mycelium segments.

37. A method for producing a mycelium-based food product comprising:
providing an aerial mycelium; and
processing the aerial mycelium in a vacuum tumbler to form a corresponding processed aerial mycelium.
